(19) United States
(12) Patent Application Publication
(10) Pub. No.: US 2020/0315894 A1
(43) Pub. Date: Oct. 8, 2020

(54) DRESSING WITH TARGETED LOW-INTENSITY VIBRATION

(71) Applicant: HILL-ROM SERVICES, INC., Batesville, IN (US)

(72) Inventors: Michael Churilla, Harrison, OH (US); Charles A. Lachenbruch, Batesville, IN (US); Frank E. Sauser, Cincinnnati, OH (US)

(21) Appl. No.: 16/838,282

(22) Filed: Apr. 2, 2020

Related U.S. Application Data
(60) Provisional application No. 62/828,683, filed on Apr. 3, 2019.

(51) Int. Cl. A61H 1/00; A61F 13/505

(52) U.S. Cl. CPC A61H 1/00; A61H 23/0245; A61F 13/505

(57) ABSTRACT

Vibrational dressing systems, vibrational dressing devices, and methods for placing the same are disclosed. Vibrational dressing systems and vibrational dressing devices include a first portion and a second portion. The first portion may be contoured to a target treatment location of a subject. The second portion may be coupled to the first portion and the second portion may include a plurality of vibrational devices and a control system. Each vibrational device may vibrate the target treatment location and each vibrational device may be positioned within the second portion to correspond with a particular location on a body of the subject within the target treatment location when the first portion is coupled to the body of the subject. The control system may selectively activate one or more of the plurality of vibrational devices.

### CROSS-REFERENCE TO RELATED APPLICATIONS

The present specification claims priority to U.S. Provisional Patent Application Ser. No. 62/828,683, filed Apr. 3, 2019, entitled “DRESSING WITH TARGETED LOW-INTENSITY VIBRATION,” the entirety of which is incorporated by reference herein.

### BACKGROUND

### Field

The present disclosure generally relates to systems, devices, and/or methods for promoting chronic wound healing, and more specifically, to systems, devices and/or methods that include a plurality of particularly located vibrational devices that provide vibrations to particular locations on a body of a subject within a target treatment location.

### Technical Background

In some medical situations, a subject may develop pressure injuries as a result of extended periods of an application of pressure on a portion of the subject's body (e.g., bedsores). If not treated properly, a pressure injury may develop into a chronic wound that can result in further complications. Accordingly, any particular portion of the subject's body that may develop a pressure injury, is developing a pressure injury, or already has developed a pressure injury must be properly treated to avoid further complications.

### SUMMARY

In one aspect, a vibrational dressing device may include a first portion and a second portion. The first portion may be contoured to a target treatment location of a subject. The second portion may be coupled to the first portion and the second portion may include a plurality of vibrational devices and a control system. Each vibrational device may vibrate the target treatment location and each vibrational device may be positioned within the second portion to correspond with a particular location on a body of the subject within the target treatment location when the first portion is coupled to the body of the subject. The control system may activate one or more of the plurality of vibrational devices. According to various aspects, the plurality of vibrational devices may include a first vibrational device, a second vibrational device, and a third vibrational device, where the first vibrational device may be located within the second portion to correspond with a first ischial tuberosity of the subject, the second vibrational device may be located within the second portion to correspond with a second ischial tuberosity of the subject, and the third vibrational device may be located within the second portion to correspond with the sacrum of the subject. In such an aspect, the first portion may be contoured to a sacrum of the subject. In yet another aspect, the plurality of vibrational devices may be arranged within the second portion as a predefined array of vibrational devices. In such an aspect, the predefined array of vibrational devices may be based on at least one distance calculated between one or more pinpoint locations of the both of the subject. In other aspects, the control system may include a vibration switch, and a signal generator that transmits a signal to the plurality of vibrational devices, the signal causing the plurality of vibrational devices to generate low-intensity vibrations. In such an aspect, the signal may cause the plurality of vibrational devices to generate vibrations having a peak acceleration from about 0.2 g to about 0.4 g. In some aspects, the control system may activate the plurality of vibrational devices according to a predefined schedule and/or intermittently. In other aspects, the control system may further include a power device, where the power device includes a wireless power receiver to wirelessly power, wirelessly charge, or wirelessly recharge the power device. In yet other aspects, the control system may further include a processor and a memory, where the memory stores program instructions executable by the processor to send a control signal to the signal generator, the control signal causing the signal generator to generate and to transmit the signal to the plurality of vibrational devices. In yet further aspects, the control system may further include a processor and a memory, where the memory stores program instructions executable by the processor to activate the plurality of vibrational devices at least one of simultaneously, individually, or intermittently. In other aspects, the vibration switch may include a pull tab. In yet other aspects, the first portion may include a bonding portion that releasably couples the first portion to the body of the subject. In such aspects, the bonding portion may extend a first width inwardly from a peripheral outer edge of the first portion around a perimeter of the first portion. Further in such aspects, the bonding portion may be offset a second width from a peripheral outer edge of the first portion.

In another aspect, a vibrational dressing device may include a first portion and a second portion. The second portion may be coupled to the first portion and the second portion may include a plurality of vibrational devices and a control system. The plurality of vibrational devices may vibrate a target treatment location and may be arranged within the second portion as a predefined array of vibrational devices. The control system may selectively activate the plurality of vibrational devices. According to various aspects, the first portion is at least one of shaped or sized to contour to the target treatment location, the target treatment location including a first ischial tuberosity of the subject, a second ischial tuberosity of the subject, and a sacrum of the subject. According to further aspects, the control system may include a vibration switch and a signal generator that transmits a signal to the plurality of vibrational devices, the signal causing the plurality of vibrational devices to generate low-intensity vibrations. In such an aspect, the signal may cause the plurality of vibrational devices to generate vibrations having a peak acceleration from about 0.2 g to about 0.4 g. In some aspects, the control device may activate the plurality of vibrational devices according to a predefined schedule and/or intermittently. In other aspects, the control system may further include a power device, the power device including a battery. Further in such an aspect, the vibration switch may include a pull tab, where after the pull tab is removed a circuit between the signal generator and the power device is established to power the plurality of vibrational devices.

In yet another aspect, a vibrational dressing system may include a disposable first portion and a reusable second portion. The reusable second portion may include a plurality of vibrational devices and a control system. Each vibrational device may be positioned within the reusable second portion to correspond with a particular location on a body of a subject within a target treatment location. The control system may include a vibration switch and a signal generator. The signal generator may transmit a signal to the plurality of vibrational devices causing the plurality of vibrational devices to generate low-intensity vibrations. According to various aspects, the first portion may be contoured to the target treatment location of the subject, the target treatment location including a first ischial tuberosity of the subject, a second ischial tuberosity of the subject, and a sacrum of the subject. According to further aspects, the plurality of vibrational devices may be arranged within the second portion as a predefined array of vibrational devices. In such an aspect, the predefined array of vibrational devices may be based on at least one distance calculated between one or more pinpoint location of the body of the subject. According to yet further aspects, the plurality of vibrational devices may include a first vibrational device, a second vibrational device, and a third vibrational device, where the first vibrational device may be located within the second portion to correspond with a first ischial tuberosity of the subject, the second vibrational device may be located within the second portion to correspond with a second ischial tuberosity of the subject, and the third vibrational device may be located within the second portion to correspond with the sacrum of the subject. In other aspects, the control system may further include a processor and a memory, where the memory stores program instructions executable by the processor to send a control signal to the signal generator, the control signal causing the signal generator to generate and to transmit the signal to the plurality of vibrational devices. In further aspects, the control system may further include a processor and a memory, where the memory stores program instructions executable by the processor to activate the plurality of vibrational devices at least one of simultaneously, individually, or intermittently. In yet other aspects, the control system may further include a battery including a wireless power receiver to wirelessly power, wirelessly charge, or wirelessly recharge the battery. According to other aspects, the signal transmitted by the signal generator may cause the plurality of vibrational devices to generate vibrations having a peak acceleration from about 0.2 g to about 0.4 g. In some aspects, the control device may activate the plurality of vibrational devices according to a predefined schedule and/or intermittently. According to yet other aspects, at least one of the disposable first portion or the reusable second portion may include a skin warming unit to selectively heat the target treatment location. In such aspects, the control system may further include a heat switch to selectively heat the target treatment location. Further in such aspects, the skin warming unit may include a coil that surrounds each of the plurality of vibrational devices to focus heat to the target treatment location.

In another aspect, a method for placing a vibrational dressing device on a subject may include determining a location of a first ischial tuberosity of the subject's body, a second ischial tuberosity of the subject's body, and a sacrum of the subject's body, and positioning a first vibrational device of the vibrational dressing over the first ischial tuberosity of the subject's body, a second vibrational device of the vibrational dressing over the second ischial tuberosity of the subject's body, and a third vibrational device of the vibrational dressing over the sacrum of the subject's body. According to various aspects, the method may further include actuating a vibration switch of the vibrational dressing device to selectively activate at least one of the first vibrational device, the second vibrational device, or the third vibrational device. In such aspects, the vibration switch may include a pull tab and actuating the vibration switch may include removing the pull tab. According to other aspects, the method may further include coupling a wireless power receiver of the vibrational dressing device with a wireless power transmitter to at least one of wirelessly power, wirelessly charge, or wirelessly recharge a power device of the vibrational dressing device.

In yet another aspect, a method for placing a vibrational dressing system on a subject may include determining a location of a first ischial tuberosity of the subject's body, a second ischial tuberosity of the subject's body, and a sacrum of the subject's body, inserting a reusable second portion into a pocket of a disposable first portion to form the vibrational dressing system, and positioning the vibrational dressing system on the subject such that a first vibrational device of the reusable second portion is located over the first ischial tuberosity of the subject's body, a second vibrational device of the reusable second portion is located over the second ischial tuberosity of the subject's body, and a third vibrational device of the reusable second portion is located over the sacrum of the subject's body. According to various aspects, the method may further include actuating a vibration switch of the reusable second portion to selectively activate at least one of the first vibrational device, the second vibrational device, or the third vibrational device. In such aspects, the vibration switch may include a pull tab and actuating the vibration switch may include removing the pull tab. According to other aspects, the method may further include coupling a wireless power receiver of the reusable second portion with a wireless power transmitter to at least one of wirelessly power, wirelessly charge, or wirelessly recharge a power device of the reusable second portion.

Additional features and advantages of the aspects described herein will be set forth in the detailed description which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1A depicts a plan view of an illustrative vibrational dressing device that applies a low-intensity vibration to a target treatment location according to one or more embodiments of the present disclosure;

FIG. 1B depicts a perspective view of the vibrational dressing device of FIG. 1A according to one or more embodiments of the present disclosure;

FIG. 2A depicts a plan view of another illustrative vibrational dressing device that applies a low-intensity vibration to a target treatment location according to one or more embodiments of the present disclosure;

FIG. 2B depicts a perspective view of the vibrational dressing device of FIG. 2A according to one or more embodiments of the present disclosure;

FIG. 3A depicts a perspective view of a reusable portion of a vibrational dressing system that applies a low-intensity vibration to a target treatment location according to one or more embodiments of the present disclosure;

FIG. 3B depicts a perspective view of a disposable portion of the vibrational dressing system that applies a low-intensity vibration to a target treatment location according to one or more embodiments of the present disclosure;

FIG. 3C depicts a plan view of the vibrational dressing system including the reusable portion of FIG. 3A removably inserted into a pocket of the disposable portion of FIG. 3B according to one or more embodiments of the present disclosure;

FIG. 4 depicts a plan view of another illustrative vibrational dressing device that applies a low-intensity vibration and heat to a target treatment location according to one or more embodiments of the present disclosure; and

FIG. 5 depicts a flow diagram of an illustrative method for placing a vibrational dressing device or a vibrational dressing system on a subject's body according to one or more embodiments of the present disclosure.

### DETAILED DESCRIPTION

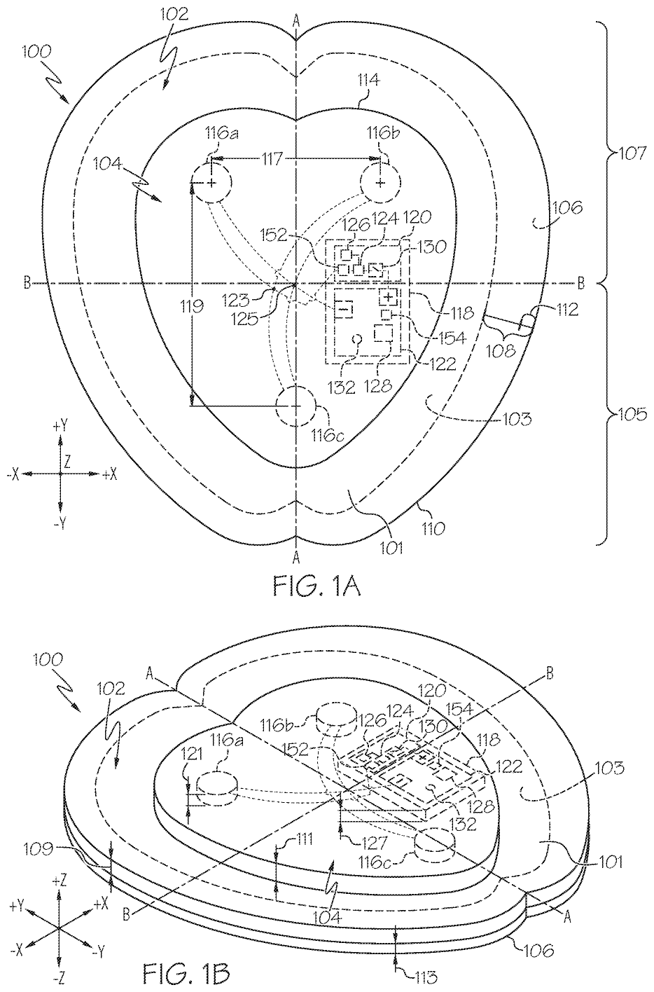

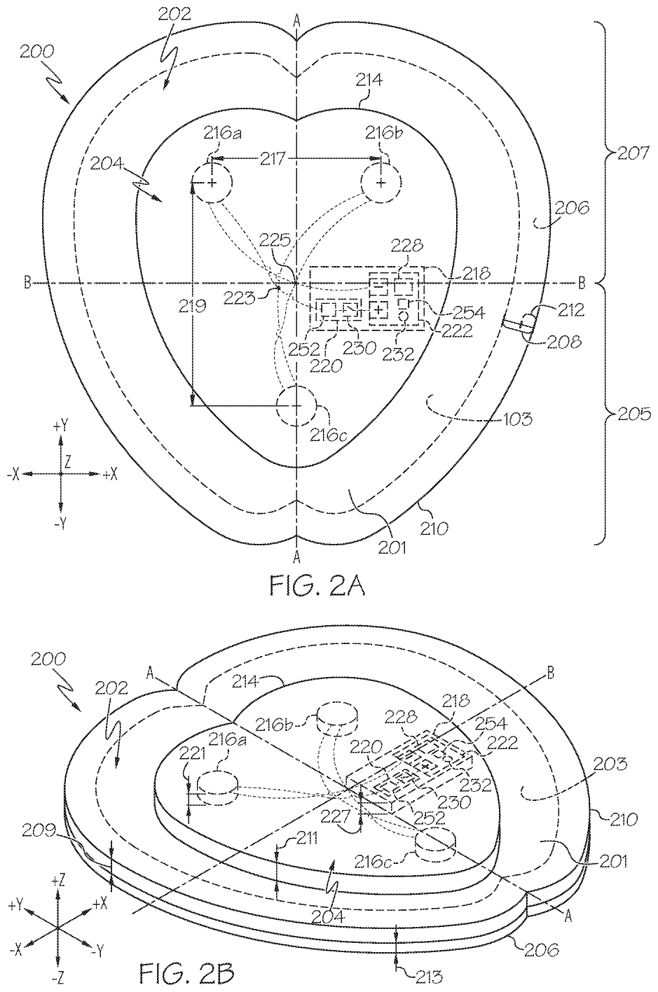

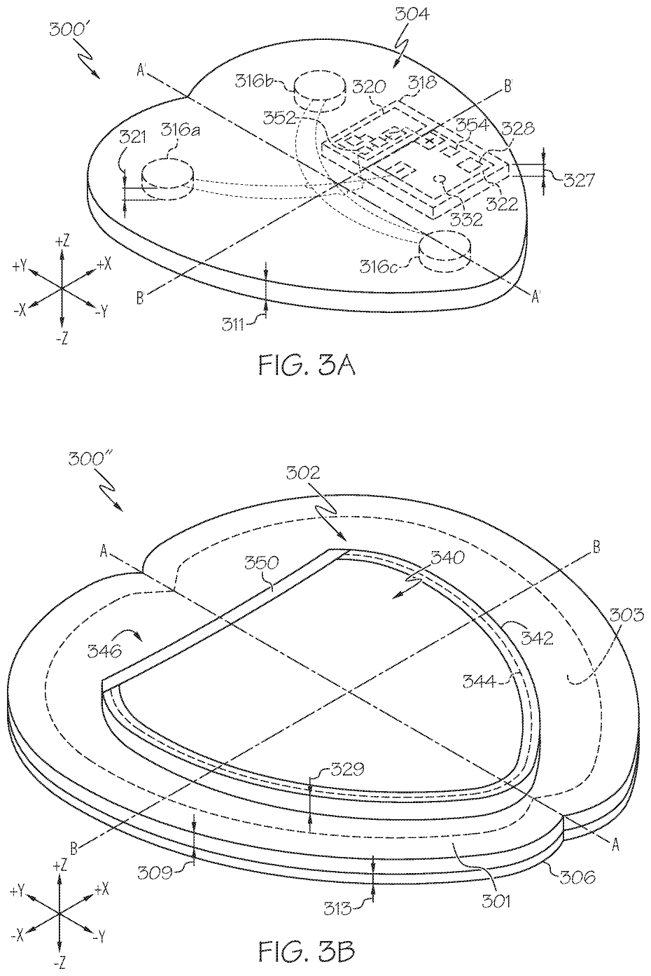

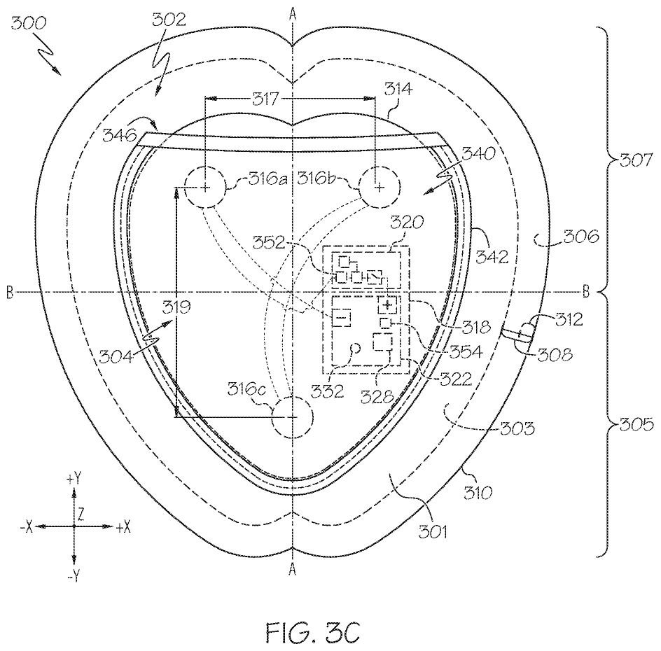

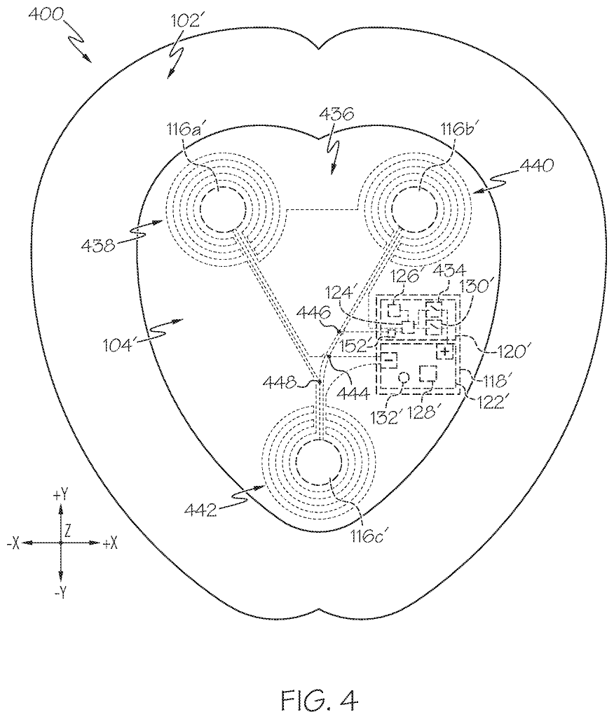

Reference will now be made in detail to embodiments of devices and/or systems that apply vibration to a target treatment location of a subject, examples of which are illustrated in the accompanying drawings. Whenever possible, the same reference numerals will be used throughout the drawings to refer to the same or like parts. Embodiments of the devices and/or systems are depicted in FIGS. 1A-1B, 2A-2B, 3A-3C, and 4. The devices and/or systems generally include a first portion configured to contour to a target treatment location of a subject and a second portion coupled to the first portion. More specifically, the second portion is either fixedly coupled to the first portion (FIGS. 1A-1B, 2A-2B, 4) or removably coupled to the first portion (FIGS. 3A-3C). Furthermore, the second portion generally includes a plurality of vibrational devices and a control system. The plurality of vibrational devices may be positioned within the second portion to correspond with a particular location on a body of the subject within the target treatment location and the control system may be configured to activate one or more of the plurality of vibrational devices. The control system generally includes a control device having a vibration switch and a signal generator. More specifically, the control device either includes a processor and a memory (FIGS. 1A-1B, 3A-3C, and 4) or does not include the processor and the memory (FIG. 2). In embodiments where the control device includes the processor and the memory, the memory stores program instructions executable by the processor such that the control device activates the plurality of vibrational devices simultaneously, substantially simultaneously, individually, and/or intermittently. In embodiments where the control device does not include the processor and the memory, the control device activates the plurality of vibrational devices simultaneously or substantially simultaneously. Furthermore, embodiments of the devices and/or systems either include a skin warming unit (FIG. 4) or do not include the skin warming unit (FIGS. 1A-1B, 2A-2B, and 3A-3C). Various embodiments of the devices and/or systems that apply vibration to a target treatment location of a subject will be described herein with specific reference to the appended drawings.

Systems, devices, and/or methods of the present disclosure may provide targeted, low-intensity vibration to areas of a subject's body at high risk for pressure injury development.

A pressure injury (PI) may be described as localized damage to the skin and/or underlying soft tissue. Such an injury may occur over a bony prominence and/or form due to the use of a medical device as a result of prolonged pressure, intense pressure and/or pressure combined with shear forces. For example, a subject may be bedridden (e.g., due to sickness, old age, obesity, and/or the like). In such an example, if the subject is lying in the same position for too long and/or a medical device is continuously attached to the subject, constant pressure on a particular area (e.g., the sacral area, area to which the medical device is attached, and/or the like) may cause a PI to occur or to form. A PI may present as intact skin or as an open ulcer and may be staged using a staging system including: Stage 1 PI (e.g., including non-blanchable erythema of intact skin), Stage 2 PI (e.g., including partial-thickness skin loss with exposed dermis), Stage 3 PI (e.g., including full-thickness skin loss) or Stage 4 PI (e.g., including full-thickness skin loss and tissue loss. A pressure injury may also be classified as an unstageable PI (e.g., including obscured full-thickness skin and tissue loss) or a deep tissue PI (e.g., including persistent non-blanchable deep red, maroon, or purple discoloration).

One technique of treating a pressure injury is to perform scheduled mobilization activities with the subject to avoid prolonged pressure on that portion of the subject's body, to avoid aggravating the pressure injury, and/or to allow the pressure injury to heal naturally. However, it may be necessary to promote the healing of pressure injuries and/or chronic wounds using a system or a device, such as those described herein.

According to aspects of the present disclosure, areas of the subject's body at high risk for such pressure injuries may include tissue surrounding the sacrum, a triangular bone formed from fused sacral vertebrae at the base of the spine between the two hip bones of the pelvis, and tissue surrounding the ischial tuberosity (ITs), a pair of bony swellings located posteriorly on the superior ramus of the ischium, which forms the lower and back portion of the hip bone. Various embodiments described herein may be used to treat pressure injuries (e.g., at such locations) that present at the various stages and classifications, as described above (e.g., Stage 1 PI, Stage 2 PI, and/or the like). More specifically, systems, devices, and/or methods of the present disclosure may provide targeted, low-intensity vibration to pressure injuries and to other types of chronic wounds (e.g., diabetic ulcers, venous ulcers, and/or the like). It should be understood that the various embodiments described herein may be used to treat other skin and/or soft tissue injuries within the spirit and scope of the present disclosure.

Various systems and/or devices of the present disclosure are configured for single use. Other systems and/or devices of the present disclosure are configured for multiple uses. Such multi-use embodiments may include a disposable portion and a reusable portion.

Turning now to the figures, FIG. 1A depicts a plan view of an illustrative vibrational dressing device 100 that applies low-intensity vibration to a target treatment location according to one or more embodiments of the present disclosure and FIG. 1B depicts a perspective view of the device 100 of FIG. 1A. Referring to both FIGS. 1A and 1B, the device 100 may include a first portion 102, a second portion 104, a plurality of vibrational devices (e.g., 116a, 116b, 116c), and a control system 118.

Referring to FIGS. 1A and 1B, according to various embodiments, the first portion 102 and the second portion 104 may be formed as a single piece (e.g., molded about the various components as described herein). According to other embodiments, the first portion 102 may be coupled to the second portion 104, or vice versa. In some aspects, the first portion 102 may be fixedly coupled to the second portion 104. In other aspects, the first portion 102 may be coupled to the second portion 104 via an adhesive. According to yet other aspects, the first portion 102 may be sewn to the second portion 104. According to further aspects, the first portion 102 may be coupled to the second portion 104 via one or more mechanical fastener (e.g., a snap, a clip, and/or the like). It should be appreciated that other manners of coupling the first portion 102 and the second portion 104 are within the spirit and scope of the present disclosure.

According to various aspects, the first portion 102 may be contoured to correspond to a target treatment location. In one example, the target treatment location may include the sacrum of a subject. In another example, the target treatment location may include the sacrum, the first ischial tuberosity of the subject, and the second ischial tuberosity of the subject. According to various embodiments, the first portion 102 may be shaped and/or sized to contour to the target treatment location. For example, in view of FIG. 1A, the first portion 102 may be generally heart-shaped. In such an aspect, a lower section 105 (e.g., section below axis B-B in the −Y direction of the coordinate axes of FIG. 1A) of the first portion 102 may be shaped and/or sized to correspond to the sacrum of the subject (e.g., relatively narrow to fit the sacrum area) and an upper section 107 (e.g., section above axis B-B in the +Y direction of the coordinate axes of FIG. 1A) of the first portion 102 may be shaped and/or sized to correspond to the ITs of the subject (e.g., relatively wide to span the IT locations, about the axis A-A in the −X direction and the +X direction of the coordinate axis of FIG. 1A). According to various aspects, the first portion 102 may be shaped and/or sized to allow a plurality of particularly-located vibrational devices (e.g., as described herein) to provide vibrations (e.g., vibrational energy) to particular locations on a body of the subject within the target treatment location (e.g., the first portion 102 may be shaped and/or sized to accommodate such particularly-located vibrational devices). In one example, the first portion 102 may be shaped and/or sized similar to a Mepilex® Border Sacrum Dressing (Molnlycke Health Care AB, Gothenburg, Sweden).

Referring to FIGS. 1A and 1B, the first portion 102 may include a first material. The first material may include a dressing material (e.g., an antimicrobial dressing, a foam dressing, and/or the like). In view of FIG. 1B, the first portion 102 may be configured to have a first portion thickness 109. The first portion 102 may include a top surface 101 and a bottom surface 103, the bottom surface 103 including a bonding portion 106 configured to releasably couple the first portion 102 to a body of the subject. In view of FIG. 1B, the bonding portion 106 may be configured to have a bond thickness 113. In some aspects, the bonding portion 106 may include a medical adhesive. Referring to FIG. 1A, the bonding portion 106 (shown in phantom) may extend a first width 108 inwardly from a peripheral outer edge 110 of the first portion 102 around a perimeter of the first portion 102. According to various aspects, the first width 108 may extend inwardly from the peripheral outer edge 110 of the first portion 102 to the peripheral outer edge 114 of the second portion 104. According to some embodiments, the first width 108 may be uniform around the perimeter of the first portion 102. According to other embodiments, the first width 108 may be non-uniform around the perimeter of the first portion 102. That is, a first section of the bonding portion 106 may have one bond width, a second section of the bonding portion 106 may have another bond width, and/or the like. For example, a relatively larger first width 108 may be located along the perimeter of the first portion 102 where a movement and/or a flexing of subject skin may occur. According to some aspects, the first width 108 may extend inwardly to define an inner portion (not shown) on the bottom surface 103, the inner portion including the first material and devoid of the bonding portion 106 (e.g., the inner portion defined to cover the target treatment location). In such aspects, the bonding portion 106 may effectively seal the inner portion to generate a positive wound environment (e.g., a moist environment ideal for wound healing, an environment that promotes a balance between growth factors and inhibitors, and/or the like). According to other aspects, the first width 108 may extend inwardly to a central position of the of the first portion 102 (e.g., such that the bonding portion 106 extends inwardly from the peripheral outer edge 110 of the first portion 102 to the central position of the first portion 102 to completely cover or nearly completely cover the bottom surface 103 of the first portion 102). According to yet further aspects, the first width 108 may be offset a second width 112 inwardly from the peripheral outer edge 110 of the first portion 102 (e.g., such that the bonding portion 106 extends inwardly, as described above, from the second width 112 in lieu of the peripheral outer edge 110, as described above).

Still referring to FIGS. 1A and 1B, the second portion 104 may include a second material. According to embodiments described herein, the second material may be configured to minimize an interface pressure between the target treatment location of the subject and components of the second portion 104 (e.g., as described herein) and/or an external surface (e.g., a chair, a hospital bed, and/or the like). For example, the second material may be configured as a padding that supports the components of the second portion 104 such that the subject may be unable to noticeably feel, detect, or observe the presence of the components when seated or lying down. In view of FIG. 1B, the second portion 104 may be configured to have a second portion thickness 111. According to various aspects, the second material may include a soft foam material. According to other aspects, the second material may include a gel material.

Referring again to FIGS. 1A and 1B, the second portion 104 may include the plurality of vibrational devices (e.g., 116a, 116b, 116c) and the control system 118. According to various embodiments, each of the plurality of vibrational devices 116a, 116b, 116c may be configured to, when activated, deliver vibrations (e.g. vibrational energy). In some aspects, each vibrational device may include an eccentric rotating mass motor. In other aspects, each vibrational device may include a piezoelectric actuator, a linear electro-magnetic actuator, a magnetostrictive actuator, an electroactive polymer and/or the like. Although only three (3) vibrational devices 116a, 116b, 116c are depicted in FIGS. 1A and 1B, it should be understood that embodiments described herein may include more than three or less than three vibrational devices. According to various aspects each vibrational device 116a, 116b, 116c may be embedded within the second portion 104 of the device 100. According to other aspects, at least a portion of each vibrational device 116a, 116b, 116c may also be embedded in the first portion 102. In some embodiments, each of the vibrational device 116a, the vibrational device 116b, and/or the vibrational device 116c may be located within the second portion 104 to interface or correspond with a specific location of or on the subject's body (e.g., within the target treatment location) when the first portion 102 is coupled to the body of the subject (e.g., via the bonding portion 106). In one aspect, the vibrational device 116a may be located to interface or correspond with the first ischial tuberosity (IT) of the subject, the vibrational device 116b may be located to interface or correspond with the second ischial tuberosity (IT) of the subject, and the vibrational device 116c may be located to interface or correspond with the sacrum of the subject (see FIGS. 1A and 1B, e.g., a “Y” array or configuration of vibrational devices 116a, 116b, 116c). In this vein, according to other aspects, a plurality of vibrational devices (e.g., 116a, 116b, 116c, and/or the like) may be configured in any predefined array of vibrational devices, where each vibrational device may be positioned or located within the second portion 104 to interface or correspond with a specific location of or on the subject's body (e.g., PI target, or likely PI target, within a target treatment location) when the first portion 102 is coupled to the body of the subject. According to such aspects, the device 100 of the present disclosure may be customized to target vibrations (e.g., low-intensity vibrations), as described herein, to specific pinpoint locations of the subject's body (e.g. joints, bones, tissue areas, and/or the like). For example, distances to or between one or more specific pinpoint locations of a subject's body may be calculated, and a device 100 (e.g., including one or more vibrational devices positioned or located within the second portion 104 to interface or correspond with the one or more specific pinpoint locations when the first portion 102 is coupled to the subject's body) may be customized for the subject. More specifically, in such an example, a first distance 117 (e.g., along the −X/+X axis of the coordinate axes of FIG. 1A, parallel to axis B-B) between vibrational device 116a and vibrational device 116b may correspond to a distance calculated between the first ischial tuberosity (IT) of the subject and the second ischial tuberosity (IT) of the subject. Similarly, in such an example, a second distance 119 (e.g., along the +Y/−Y axis of the coordinate axes of FIG. 1A, parallel to axis A-A) between the vibrational devices 116a, 116b and the vibrational device 116c may correspond to a distance calculated between the first and second ischial tuberosity (ITs) and the sacrum of the subject. Similarly, according to another example, average distances to or between one or more specific pinpoint locations of a population of subject bodies (e.g., gender-based, height-based, weight-based, ethnicity-based, and/or the like) may be calculated, and a device 100 (e.g., including one or more vibrational devices positioned or located within the second portion 104 to interface or correspond with the one or more specific pinpoint locations when the first portion 102 is coupled to a subject's body) may be customized for a subject within that population of subjects. According to various embodiments described herein, each vibrational device 116a, 116b, 116c may be a low-profile vibrational motor. In view of FIG. 1B, low-profile may correspond to a thickness 121 capable of being absorbed or padded by the second material of the second portion 104 and/or the first material of the first portion 102. For example, the thickness 121 of each vibrational device 116a, 116b, 116c may be less than the second portion thickness 111 and/or the first portion thickness 109. According to other aspects, low-profile may correspond to a thickness 121 such that a subject may be unable to noticeably feel, detect, or observe the presence of each vibrational device (e.g., 116a, 116b, 116c) when seated or lying down. Such a low-profile may minimize and/or avoid unnecessary pressure points within the target treatment location and/or enable a low-profile device 100 (e.g., a low-profile sacral dressing). According to various embodiments of the present disclosure, each vibrational device 116a, 116b, 116c may be configured to, when activated, provide vibrations (e.g., vibrational energy), as described herein, through the second material of the second portion 104 and/or the first material of the first portion 102 to the target treatment location (e.g., the sacrum, ITs, and/or the like) to promote increased perfusion and an increased rate of healing.

Referring again to FIGS. 1A and 1B, the control system 118 of the second portion 104 may include a control device 120 and a power device 122. The control device 120 may be electrically coupled to the power device 122 (FIG. 1A, e.g., via a wire or the like). In such aspects, the control device 120 may be configured (e.g., via firmware, hardware, software, and/or the like) to activate each vibrational device 116a, 116b, 116c. In view of FIG. 1A, according to various aspects, the control device 120 may be electrically coupled, via wire terminal 123, to vibrational device 116a, vibrational device 116b, and vibrational device 116c (e.g., wires depicted as dashed lines in FIGS. 1A and 1B). In such aspects, the control device 120 may be configured to activate all vibrational devices 116a, 116b, 116c simultaneously, substantially simultaneously, and/or intermittently. According to an alternative aspect, the control device 120 may be electrically coupled to each vibrational device 116a, 116b, 116c individually (not shown). In such an aspect, the control device 120 may be configured to activate each vibrational device 116a, 116b, 116c simultaneously, substantially simultaneously, intermittently, and/or individually. According to various aspects described herein, the control device 120 may include a vibration switch device 130, a processor 124, a memory 126, and a signal generator 152 (e.g., as described herein). The memory 126 may store program instructions executable by the processor 124 to activate each vibrational device 116a, 116b, 116c simultaneously, substantially simultaneously, individually, and/or intermittently, as described herein.

According to various aspects, a first terminal (e.g., negative terminal) of the power device 122 may be electrically coupled, via wire terminal 125, to vibrational device 116a, vibrational device 116b, and vibrational device 116c (e.g., wires depicted as dashed lines in FIGS. 1A and 1B), while a second terminal (e.g., positive terminal) of the power device 122 may be electrically coupled to the vibration switch device 130. In such an aspect, a circuit between the processor 124 and the power device 122 may be selectively interrupted by the vibration switch device 130. According to various aspects, the vibration switch device 130 may include a pull tab, where after the pull tab is removed the circuit between the processor 124 and the power device 222 is established to activate each vibrational device 116a, 116b, 116c. According to other aspects, the vibration switch device 130 may include a mechanical switch (e.g., a slide switch, on/off switch, and/or the like) configured to selectively establish, disconnect, and/or re-establish the circuit between the processor 124 and the power device 122. According to numerous aspects, the vibration switch device 130 may be externally accessible on or within the second portion 104. For example, in one embodiment (e.g., a disposable device) of the present disclosure, a user may remove a pull tab to activate (e.g., via the processor 124 executing the program instructions) each vibrational device 116a, 116b, 116c (e.g., on demand) before, during, or after the device 100 is applied to the target treatment location. In another example, a user may selectively activate (e.g., via the processor 124 executing the program instructions) each vibrational device 116a, 116b, 116c by actuating the vibration switch device 130 (e.g., to an “on” position). According to various aspects, the vibration switch device 130 may be configured to avoid an accidental actuation (e.g., from the “off” to the “on” position and/or from the “on” to the “off” position). In some aspects, the vibration switch device 130 may include a locking mechanism (not shown) configured to lock the vibration switch device 130 in the “on” position and/or the “off” position. In other aspects, the vibration switch device 130 may be configured such that it is not actuated by an interface pressure (e.g., due to subject weight) between the second portion 104 (e.g., and/or components thereof) and the target treatment location of the subject and/or an external surface (e.g., a chair, a hospital bed, and/or the like).

Further in such aspects, the power device 122 may be configured to power the plurality of vibrational devices (e.g., 116a, 116b, 116c). Such a power device 122 may be positioned within the second portion 104 at a predetermined location that avoids spatial interference with the plurality of vibrational devices (e.g., to minimize and/or avoid unnecessary pressure points within the target treatment location and/or to enable a low-profile device 100). The power device 122 may have a low-profile. Referring to FIG. 1B, low-profile may correspond to a thickness 127 capable of being absorbed or padded by the second material of the second portion 104 and/or the first material of the first portion 102. For example, the thickness 127 of the power device 122 may be less than the second portion thickness 111 and/or the first portion thickness 109. According to other aspects, low-profile may correspond to a thickness 127 such that a subject may be unable to noticeably feel, detect, or observe the presence of the power device 122 when seated or lying down. According to various embodiments, the power device 122 may be configured to power the plurality of vibrational devices for a predetermined period of time. According to some embodiments, the power device 122 may include a wireless power receiver 128 configured to wirelessly power, wirelessly charge, and/or wirelessly recharge the power device 122. In one example, the wireless power receiver 128 may include a reception area having electromagnetic receiving portion (not shown). According to such aspects, the reception area of the wireless power receiver 128 may be placed in proximity to and/or in alignment with an active area of an electromagnetic emitting portion (e.g., inductive coils generating an electromagnetic field) of a wireless power transmitter (not shown) to transfer power to and wirelessly power, wirelessly charge, and/or wirelessly recharge the power device 122. According to further embodiments, the power device 122 may include an externally accessible power port 132 to power, charge, and/or recharge the power device 122 (e.g., via an AC to DC power supply, and/or the like). According to various aspects, the power device 122 may include a battery. According to some aspects, the power device 122 may include a rechargeable battery. According to alternative aspects, the power device 122 may include an externally accessible power port 132 to power the plurality of vibrational devices (e.g., 116a, 116b, 116c) with an external power source when a power source internal to the second portion 104 is not present or is depleted. According to some aspects, the power device 122 may include an alert device 154 (e.g., beeper, indicator light, wireless transmitter, and/or the like) configured to signal (e.g., audibly, visually, wirelessly, and/or the like) that the power device 122 is depleted or near depletion. For example, the alert device 154 (e.g., wireless transmitter) may transmit a signal to a wireless receiver (e.g., a cell phone, a user/subject device, a caregiver device, and/or the like) that the power device 122 is depleted or near depletion (e.g., to prompt replacement of the sacral dressing, to prompt a charge/recharge of the power device 122, and/or the like).

FIG. 2A depicts a plan view of another illustrative vibrational dressing device 200 that applies a low-intensity vibration to a target treatment location according to one or more embodiments of the present disclosure and FIG. 2B depicts a perspective view of the device 200 of FIG. 2A. Referring to both FIGS. 2A and 2B, the device 200 may include a first portion 202, a second portion 204, a plurality of vibrational devices (e.g., 216a, 216b, 216c), and a control system 218.

Referring to FIGS. 2A and 2B, according to various embodiments, the first portion 202 and the second portion 204 may be formed as a single piece (e.g., molded about the various components as described herein). According to other embodiments, the first portion 202 may be coupled to the second portion 204, or vice versa. In some aspects, the first portion 202 may be fixedly coupled to the second portion 204. In other aspects, the first portion 202 may be coupled to the second portion 204 via an adhesive. According to yet other aspects, the first portion 202 may be sewn to the second portion 204. According to further aspects, the first portion 202 may be coupled to the second portion 204 via one or more mechanical fastener (e.g., a snap, a clip, and/or the like). It should be appreciated that other manners of coupling the first portion 202 and the second portion 204 are within the spirit and scope of the present disclosure.

Similar to the device 100 of FIGS. 1A and 1B, the first portion 202 of device 200 may be contoured to correspond to a target treatment location. In one example, the target treatment location may include the sacrum of a subject. In another example, the target treatment location may include the sacrum, the first ischial tuberosity of the subject, and the second ischial tuberosity of the subject. According to various embodiments, the first portion 202 may be shaped and/or sized to contour to the target treatment location. For example, in view of FIG. 2A, the first portion 202 may be generally heart-shaped. In such an aspect, a lower section 205 (e.g., section below axis B-B in the −Y direction of the coordinate axes of FIG. 2A) of the first portion 202 may be shaped and/or sized to correspond to the sacrum of the subject (e.g., relatively narrow to fit the sacrum area) and an upper section 207 (e.g., section above axis B-B in the +Y direction of the coordinate axes of FIG. 2A) of the first portion 202 may be shaped and/or sized to correspond to the ITs of the subject (e.g., relatively wide to span the IT locations, about the axis A-A in the −X direction and the +X direction of the coordinate axis of FIG. 2A). According to various aspects, the first portion 202 may be shaped and/or sized to allow a plurality of particularly-located vibrational devices (e.g., as described herein) to provide vibrations (e.g., vibrational energy) to particular locations on a body of the subject within the target treatment location (e.g., the first portion 202 may be shaped and/or sized to accommodate such particularly-located vibrational devices). In one example, the first portion 202 may be shaped and/or sized similar to a Mepilex® Border Sacrum Dressing (Molnlycke Health Care AB, Gothenburg, Sweden).

Referring to FIGS. 2A and 2B, the first portion 202 may include a first material. The first material may include a dressing material (e.g., an antimicrobial dressing, a foam dressing, and/or the like). In view of FIG. 2B, the first portion 202 may be configured to have a first portion thickness 209. The first portion 202 may include a top surface 201 and a bottom surface 203, the bottom surface 203 including a bonding portion 206 configured to releasably couple the first portion 202 to a body of the subject. In view of FIG. 2B, the bonding portion 206 may be configured to have a bond thickness 213. In some aspects, the bonding portion 206 may include a medical adhesive. Referring to FIG. 2A, the bonding portion 206 (shown in phantom) may extend a first width 208 inwardly from a peripheral outer edge 210 of the first portion 202 around a perimeter of the first portion 202. According to various aspects, the first width 208 may extend inwardly from the peripheral outer edge 210 of the first portion 202 to the peripheral outer edge 214 of the second portion 204. According to some embodiments, the first width 208 may be uniform around the perimeter of the first portion 202. According to other embodiments, the first width 208 may be non-uniform around the perimeter of the first portion 202. That is, a first section of the bonding portion 206 may have one bond width, a second section of the bonding portion 206 may have another bond width, and/or the like. For example, a relatively larger first width 208 may be located along the perimeter of the first portion 202 where a movement and/or a flexing of subject skin may occur. According to some aspects, the first width 208 may extend inwardly to define an inner portion (not shown) on the bottom surface 203, the inner portion including the first material and devoid of the bonding portion 206 (e.g., the inner portion defined to cover the target treatment location). In such aspects, the bonding portion 206 may effectively seal the inner portion to generate a positive wound environment (e.g., a moist environment ideal for wound healing, an environment that promotes a balance between growth factors and inhibitors, and/or the like). According to other aspects, the first width 208 may extend inwardly to a central position of the of the first portion 202 (e.g., such that the bonding portion 206 extends inwardly from the peripheral outer edge 210 of the first portion 202 to the central position of the first portion 202 to completely cover or nearly completely cover the bottom surface 203 of the first portion 202). According to yet further aspects, the first width 208 may be offset a second width 212 inwardly from the peripheral outer edge 210 of the first portion 202 (e.g., such that the bonding portion 206 extends inwardly, as described above, from the second width 212 in lieu of the peripheral outer edge 210, as described above).

Still referring to FIGS. 2A and 2B, the second portion 204 may include a second material. According to embodiments described herein, the second material may be configured to minimize an interface pressure between the target treatment location of the subject and components of the second portion 204 (e.g., as described herein) and/or an external surface (e.g., a chair, a hospital bed, and/or the like). For example, the second material may be configured as a padding that supports the components of the second portion 204 such that the subject may be unable to noticeably feel, detect, or observe the presence of the components when seated or lying down. In view of FIG. 2B, the second portion 204 may be configured to have a second portion thickness 211. According to various aspects, the second material may include a soft foam material. According to other aspects, the second material may include a gel material.

Referring again to FIGS. 2A and 2B, similar to the device 100 of FIGS. 1A and 1B, the second portion 204 may include the plurality of vibrational devices (e.g., 216a, 216b, 216c) and the control system 218. According to various embodiments, each of the plurality of vibrational devices 216a, 216b, 216c may be configured to, when activated, deliver vibrations (e.g., vibrational energy). In some aspects, each vibrational device may include an eccentric rotating mass motor. In other aspects, each vibrational device may include a piezoelectric actuator, a linear electro-magnetic actuator, a magnetostrictive actuator, an electroactive polymer and/or the like. Although only three (3) vibrational devices 216a, 216b, 216c are depicted in FIGS. 2A and 2B, it should be understood that embodiments described herein may include more than three or less than three vibrational devices. According to various aspects each vibrational device 216a, 216b, 216c may be embedded within the second portion 204 of the device 200. According to other aspects, at least a portion of each vibrational device 216a, 216b, 216c may also be embedded within the first portion 202. In some embodiments, each of the vibrational device 216a, the vibrational device 216b, and/or the vibrational device 216c may be located within the second portion 204 to interface or correspond with a specific location of or on the subject's body (e.g., within the target treatment location) when the first portion 202 is coupled to the body of the subject (e.g., via the bonding portion 206). In one aspect, the vibrational device 216a may be located to interface or correspond with the first ischial tuberosity (IT) of the subject, the vibrational device 216b may be located to interface or correspond with the second ischial tuberosity (IT) of the subject, and the vibrational device 216c may be located to interface or correspond with the sacrum of the subject (see FIG. 2A, e.g., a “Y” array or configuration of vibrational devices 216a, 216b, 216c). In this vein, according to other aspects, a plurality of vibrational devices (e.g., 216a, 216b, 216c, and/or the like) may be configured in any predefined array of vibrational devices, where each vibrational device may be positioned or located within the second portion 204 to interface or correspond with a specific location of or on the subject's body (e.g., PI target, or likely PI target, within the target treatment location) when the first portion 202 is coupled to the body of the subject. According to such aspects, the device 200 of the present disclosure may be customized to target vibrations (e.g., low-intensity vibrations), as described herein, to specific pinpoint locations of the subject's body (e.g., pressure injury areas, joints, bones, and/or the like). For example, distances to or between one or more specific pinpoint locations of a subject's body may be calculated, and a device 200 (e.g., including one or more vibrational devices positioned or located within the second portion 204 to interface or correspond with the one or more specific pinpoint locations when the first portion 202 is coupled to the subject's body) may be customized for the subject. More specifically, in such an example, a first distance 217 (e.g., along the −X/+X axis of the coordinate axes of FIG. 2A, parallel to axis B-B) between vibrational device 216a and vibrational device 216b may correspond to a distance calculated between the first ischial tuberosity (IT) of the subject and the second ischial tuberosity (IT) of the subject. Similarly, in such an example, a second distance 219 (e.g., along the +Y/−Y axis of the coordinate axes of FIG. 2A, parallel to axis A-A) between the vibrational devices 116a, 116b and the vibrational device 116c may correspond to a distance calculated between the first and second ischial tuberosity (ITs) and the sacrum of the subject. Similarly, according to another example, average distances to or between one or more specific pinpoint locations of a population of subject bodies (e.g., gender-based, height-based, weight-based, ethnicity-based, and/or the like) may be calculated, and a device 200 (e.g., including one or more vibrational devices positioned or located within the second portion 204 to interface or correspond with the one or more specific pinpoint locations when the first portion 202 is coupled to a subject's body) may be customized for a subject within that population of subjects. According to various embodiments described herein, each vibrational device 216a, 216b, 216c may be a low-profile vibrational motor. In view of FIG. 2B, low-profile may correspond to a thickness 221 capable of being absorbed or padded by the second material of the second portion 204 and/or the first material of the first portion 202. For example, the thickness 221 of each vibrational device 216a, 216b, 216c may be less than the second portion thickness 211 and/or the first portion thickness 209. According to other aspects, low-profile may correspond to a thickness 221 such that a subject may be unable to noticeably feel, detect, or observe the presence of each vibrational device (e.g., 216a, 216b, 216c) when seated or lying down. Such a low-profile may minimize and/or avoid unnecessary pressure points within the target treatment location and/or enable a low-profile device 200 (e.g., a low-profile sacral dressing). According to various embodiments of the present disclosure, each vibrational device 216a, 216b, 216c may be configured to, when activated, provide vibrations (e.g., vibrational energy), as described herein, through the second material of the second portion 204 and/or the first material of the first portion 202 to the target treatment location (e.g., the sacrum, ITs, and/or the like) to promote increased perfusion and an increased rate of healing.

Referring again to FIGS. 2A and 2B, the control system 218 of the second portion 204 may include a control device 220 and a power device 222. The control device 220 may be electrically coupled to the power device 222 (e.g., via a wire or the like). In such aspects, the control device 220 may be configured to activate each vibrational device 216a, 216b, 216c. In view of FIG. 2A, according to various aspects, the control device 220 may be electrically coupled, via wire terminal 223, to vibrational device 216a, vibrational device 216b, and vibrational device 216c (e.g., wires depicted as dashed lines in FIGS. 2A and 2B). In such aspects, the control device 220 may be configured to activate all vibrational devices 216a, 216b, 216c simultaneously or substantially simultaneously. In the device 200 of FIG. 2A, the control device 220 may include a vibration switch device 230 and a signal generator 252 (e.g., as described herein).

According to various aspects, a first terminal (e.g., negative terminal) of the power device 222 may be electrically coupled, via wire terminal 225, to vibrational device 216a, vibrational device 216b, and vibrational device 216c (e.g., wires depicted as dashed lines in FIGS. 2A and 2B), while a second terminal (e.g., positive terminal) of the power device 222 may be electrically coupled to the vibration switch device 230. In such an aspect, a circuit between the signal generator 252 and the power device 222 may be selectively interrupted by the vibration switch device 230. According to various aspects, the vibration switch device 230 may include a pull tab, where after the pull tab is removed the circuit between the signal generator 252 and the power device 222 is established to power or activate each vibrational device 216a, 216b, 216c simultaneously or substantially simultaneously. According to other aspects, the vibration switch device 230 may include a mechanical switch (e.g., a slide switch, on/off switch, and/or the like) configured to selectively establish, disconnect and/or re-establish the circuit between the signal generator 252 and the power device 222. According to numerous aspects the vibration switch device 230 may be externally accessible on or within the second portion 204. For example, in one embodiment (e.g., a disposable device) of the present disclosure, a user may remove a pull tab to activate each vibrational device 216a, 216b, 216c (e.g., on demand) before, during, or after the device 200 is applied to the target treatment location. In another example, a user may selectively power each vibrational device 216a, 216b, 216c by actuating the vibration switch device 230 (e.g., to an “on” position). According to various aspects, the vibration switch device 230 may be configured to avoid an accidental actuation (e.g., from the “off” to the “on” position and/or from the “on” to the “off” position). In some aspects, the vibration switch device 230 may include a locking mechanism (not shown) configured to lock the vibration switch device 230 in the “on” position and/or the “off” position. In other aspects, the vibration switch device 230 may be configured such that it is not actuated by an interface pressure (e.g., due to subject weight) between the second portion 204 (e.g., and/or components thereof) and the target treatment location of the subject and/or an external surface (e.g., a chair, a hospital bed, and/or the like).

Further in such aspects, the power device 222 may be configured to power the plurality of vibrational devices (e.g., 216a, 216b, 216c). Such a power device 222 may be positioned within the second portion 204 at a predetermined location that avoids spatial interference with the plurality of vibrational devices (e.g., to minimize and/or avoid unnecessary pressure points within the target treatment location and/or to enable a low-profile device 100). The power device 222 may have a low-profile. Referring to FIG. 2B, low-profile may correspond to a thickness 227 capable of being absorbed or padded by the second material of the second portion 204 and/or the first material of the first portion 202. For example, the thickness 227 of the power device 122 may be less than the second portion thickness 211 and/or the first portion thickness 209. According to other aspects, low-profile may correspond to a thickness 227 such that a subject may be unable to noticeably feel, detect, or observe the presence of the power device 222 when seated or lying down. According to various embodiments, the power device 222 may be configured to power the plurality of vibrational devices for a predetermined period of time. According to some embodiments, the power device 222 may include a wireless power receiver 228 configured to wirelessly power, wirelessly charge, and/or wirelessly recharge the power device 222. In one example, the wireless power receiver 228 may include a reception area having electromagnetic receiving portion (not shown). According to such aspects, the reception area of the wireless power receiver 228 may be placed in proximity to and/or in alignment with an active area of an electromagnetic emitting portion (e.g., inductive coils generating an electromagnetic field) of a wireless power transmitter (not shown) to transfer power to and wirelessly power, wirelessly charge, and/or wirelessly recharge the power device 222. According to further embodiments, the power device 222 may include an externally accessible power port 232 to power, charge, and/or recharge the power device 222 (e.g., via an AC to DC power supply, and/or the like). According to various aspects, the power device 222 may include a battery. According to some aspects, the power device 222 may include a rechargeable battery. According to alternative aspects, the power device 222 may include an externally accessible power port 232 to power the plurality of vibrational devices (e.g., 216a, 216b, 216c) with an external power source when a power source internal to the second portion 204 is not present or is depleted. According to some aspects, the power device 222 may include an alert device 254 (e.g., beeper, indicator light, wireless transmitter, and/or the like) configured to signal (e.g., audibly, visually, wirelessly, and/or the like) that the power device 222 is depleted or near depletion. For example, the alert device 254 (e.g., wireless transmitter) may transmit a signal to a wireless receiver (e.g., a cell phone, a user/subject device, a caregiver device, and/or the like) that the power device 222 is depleted or near depletion (e.g., to prompt replacement of the sacral dressing, to prompt a charge/recharge of the power device 222, and/or the like).

FIG. 3A depicts a perspective view of a reusable portion 300′ of a vibrational dressing system 300 that applies a low-intensity vibration to a target treatment location, FIG. 3B depicts a perspective view of a disposable portion 300″ of the vibrational dressing system 300 that applies a low-intensity vibration to a target treatment location, and FIG. 3C depicts a plan view of the vibrational dressing system 300 including the reusable portion 300′ removably inserted into the pocket of the disposable portion 300″, according to one or more embodiments of the present disclosure. Referring to FIG. 3C, similar to the device 100 of FIGS. 1A and 1B and the device 200 of FIGS. 2A and 2B, the system 300 may include the disposable portion 300″ (FIG. 3B) having a first portion 302 and the reusable portion 300′ (FIG. 3A) having a second portion 304, the second portion 304, a plurality of vibrational devices (e.g., 316a, 316b, 316c), and a control system 318. Viewing FIG. 3C in light of FIGS. 3A and 3B, the second portion 304 of FIG. 3A may be removably coupled to the first portion 302 of FIG. 3B.

Referring to FIG. 3B, similar to the device 100 of FIGS. 1A and 1B and the device 200 of FIGS. 2A and 2B, the first portion 302 may include a first material. The first material may include a dressing material (e.g., an antimicrobial dressing, a foam dressing, and/or the like). In view of FIG. 3B, the first portion 302 may be configured to have a first portion thickness 309. However, contrary to the device 100 of FIGS. 1A and 1B and the device 200 of FIGS. 2A and 2B, the first portion 302 may define a pocket 340 configured to removably couple the second portion 304 to the first portion 302. Referring to FIG. 3B, according to various embodiments, the pocket 340 may be shaped, sized, and/or located to releasably hold the second portion 304 in a defined position and/or orientation. More specifically, the pocket 340 may be shaped, sized and/or located such that axes A′-A′ and B′-B′ of the second portion 304 align or substantially align with axes A-A and B-B of the first portion 302 when the second portion 304 is removably inserted within the pocket 340. According to some aspects, a shape and/or a size of the pocket 340 may be defined to mimic all or a portion of a shape and/or size of the second portion 304 (FIG. 3A, e.g., heart-shaped) and the pocket 340 may be located on the first portion 302 to position and/or to orient the second portion 304 (e.g., when inserted in the pocket 340) in a predefined manner (e.g., with respect to the target treatment location, with respect to a specific location of or on the subject's body, and/or the like).

Referring to FIG. 3B, the first portion 302 may be contoured to correspond to a target treatment location. In one example, the target treatment location may include the sacrum of a subject. In another example, the target treatment location may include the sacrum, the first ischial tuberosity of the subject, and the second ischial tuberosity of the subject. According to various embodiments, the first portion 302 may be shaped and/or sized to contour to the target treatment location. For example, referring to FIG. 3C, the first portion 302 may be generally heart-shaped. In such an aspect, a lower section 305 (e.g., section below axis B-B in the −Y direction of the coordinate axes of FIG. 3C) of the first portion 302 may be shaped and/or sized to correspond to the sacrum of the subject (e.g., relatively narrow to fit the sacrum area) and an upper section 307 (e.g., section above axis B-B in the +Y direction of the coordinate axes of FIG. 3C) of the first portion 302 may be shaped and/or sized to correspond to the ITs of the subject (e.g., relatively wide to span the IT locations, about the axis A-A in the −X direction and the +X direction of the coordinate axis of FIG. 3C). According to various aspects described herein, the first portion 302 of the disposable portion 300″ may be shaped and/or sized to allow a plurality of particularly-located vibrational devices (e.g., associated with the second portion 304 of the inserted reusable portion 300′) to provide vibrations (e.g., vibrational energy) to particular locations on a body of the subject within the target treatment location (e.g., the first portion 302 may be shaped and/or sized to accommodate such particularly located vibrational devices). In one example, the first portion 302 may be shaped and/or sized similar to a Mepilex® Border Sacrum Dressing (Molnlycke Health Care AB, Gothenburg, Sweden).

Referring again to FIG. 3B, the pocket 340 defined on or by the first portion 302 may also include the first material. In view of FIG. 3B, the pocket 340 may be configured to have a pocket thickness 329. In other aspects, the pocket 340 defined on or by the first portion 302 may include a material different than the first material. In one example, the pocket 340 may include a different material (e.g. an elastic and/or securing material) and the pocket 340 may be defined by coupling the different material of the pocket 340 to the first material of the first portion 302. According to various aspects, such a coupling may include sewing the first material or the different material of the pocket 340 to the first material of the first portion 302 along a coupling portion 344. According to other aspects, such a coupling may include adhering the first material or the different material of the pocket 340 to the first material of the first portion 302 (e.g., via an adhesive) along the coupling portion 344. Notably, in view of FIG. 3B, according to various aspects of the present disclosure, the pocket 340 may define an opening 346 configured for insertion of the second portion 304 of FIG. 3A. According to various aspects, the material of the pocket 340 (e.g., first material, different material, and/or the like) may include a band of material 350 at the opening 346. In such aspects, the band of material 350 (e.g., elastic band or the like) may be configured to hold the second portion 304 in the pocket 340 when the second portion 304 is inserted within the pocket 340.

Referring to FIG. 3C, similar to the device 100 of FIG. 1B and the device 200 of FIG. 2B, the first portion 302 may include a top surface 301 and a bottom surface 303, the bottom surface 303 including a bonding portion 306 configured to releasably couple the first portion 302 to a body of the subject. In view of FIG. 3B, the bonding portion 306 may be configured to have a bond thickness 313. In some aspects, the bonding portion 306 may include a medical adhesive. Referring again to FIG. 3C, the bonding portion 306 (shown in phantom) may extend a first width 308 inwardly from a peripheral outer edge 310 of the first portion 302 around a perimeter of the first portion 302. According to various aspects, the first width 308 may extend inwardly from the peripheral outer edge 310 of the first portion 302 to the peripheral outer edge 342 of the pocket 340. According to some embodiments, the first width 308 may be uniform around the perimeter of the first portion 302. According to other embodiments, the first width 308 may be non-uniform around the perimeter of the first portion 302. That is, a first section of the bonding portion 306 may have one bond width, a second section of the bonding portion 306 may have another bond width, and/or the like. For example, a relatively larger first width 308 may be located along the perimeter of the first portion 302 where a movement and/or a flexing of subject skin may occur. According to some aspects, the first width 308 may extend inwardly to define an inner portion (not shown) on the bottom surface 303, the inner portion including the first material and devoid of the bonding portion 306 (e.g., the inner portion defined to cover the target treatment location). In such aspects, the bonding portion 306 may effectively seal the inner portion to generate a positive wound environment (e.g., a moist environment ideal for wound healing, an environment that promotes a balance between growth factors and inhibitors, and/or the like). According to other aspects, the first width 308 may extend inwardly to a central position of the of the first portion 302 (e.g., such that the bonding portion 306 extends inwardly from the peripheral outer edge 310 of the first portion 302 to the central position of the first portion 302 to completely cover or nearly completely cover the bottom surface 303 of the first portion 302). According to yet further aspects, the first width 308 may be offset a second width 312 inwardly from the peripheral outer edge 310 of the first portion 302 (e.g., such that the bonding portion 306 extends inwardly, as described above, from the second width 312 in lieu of the peripheral outer edge 310, as described above).

Referring to FIG. 3A, the second portion 304 may include a second material. According to embodiments described herein, the second material may be configured to minimize an interface pressure between the target treatment location of the subject and components of the second portion 304 (e.g., as described herein) and/or an external surface (e.g., a chair, a hospital bed, and/or the like). In one example, the second material may be configured as a padding that supports the components of the second portion 304 such that the subject may be unable to noticeably feel, detect, or observe the presence of the components when seated or lying down. In view of FIG. 3A, the second portion 304 may be configured to have a second portion thickness 311. According to various aspects, the second material may include a soft foam material. According to other aspects, the second material may include a gel material. According to yet further aspects, as a reusable portion 300′, the second material may include a polymer material. In such aspects, the first material of the first portion 302 and/or the first material or different material of the pocket 340 of the first portion 302 of the disposable portion 300″ (FIG. 3B) may be configured as padding that supports the components of the second portion 304 such that the subject may be unable to noticeably feel, detect, or observe the components and/or the polymer material when seated or lying down.

In view of FIG. 3A, similar to the device 100 of FIGS. 1A and 1B and the device 200 of FIGS. 2A and 2B, the second portion 304 may include the plurality of vibrational devices (e.g., 316a, 316b, 316c) and the control system 318. According to various embodiments, each of the plurality of vibrational devices 316a, 316b, 316c may be configured to, when activated, deliver vibrations (e.g., vibrational energy). In some aspects, each vibrational device may include an eccentric rotating mass motor. In other aspects, each vibrational device may include a piezoelectric actuator, a linear electro-magnetic actuator, a magnetostrictive actuator, an electroactive polymer and/or the like. Although only three (3) vibrational devices 316a, 316b, 316c are depicted in FIG. 3A, it should be understood that embodiments described herein may include more than three or less than three vibrational devices. According to various aspects each vibrational device 316a, 316b, 316c may be embedded within the second portion 304. In some embodiments, each of the vibrational device 316a, the vibrational device 316b, and/or the vibrational device 316c may be located within the second portion 304 to interface or correspond with a specific location of or on the subject's body (e.g., within the target treatment location) when the axes A′-A′ and B′-B′ of the second portion 304 align or substantially align with the axes A-A and B-B of the first portion 302 (e.g., when the second portion 304 is inserted within the pocket 340) and when the first portion 302 is coupled to the body of the subject (e.g., via the bonding portion 306). In one aspect, the vibrational device 316a may be located to interface or correspond with the first ischial tuberosity (IT) of the subject, the vibrational device 316b may be located to interface or correspond with the second ischial tuberosity (IT) of the subject, and the vibrational device 316c may be located to interface or correspond with the sacrum of the subject (see FIG. 3C, e.g., a “Y” array or configuration of vibrational devices 316a, 316b, 316c). In this vein, according to other aspects, a plurality of vibrational devices (e.g., 316a, 316b, 316c, and/or the like) may be configured in any predefined array of vibrational devices, where each vibrational device may be positioned or located within the second portion 304 to interface or correspond with a specific location of or on the subject's body (e.g., PI target, or likely PI target, within the target treatment location) when the axes A′-A′ and B′-B′ of the second portion 304 align or substantially align with the axes A-A and B-B of the first portion 302 (e.g., when the second portion 304 is inserted within the pocket 340) and when the first portion 302 is coupled to the body of the subject. According to such aspects, a system 300 including the reusable portion 300′ and the disposable portion 300″ of the present disclosure may be customized to target vibrations (e.g., low-intensity vibrations), as described herein, to specific pinpoint locations of the subject's body (e.g., pressure injury areas, joints, bones, and/or the like). For example, distances to or between one or more specific pinpoint locations of a subject's body may be calculated, and a second portion 304, including one or more vibrational devices positioned or located within the second portion 304 to interface or correspond with the one or more specific pinpoint locations when the axes A′-A′ and B′-B′ of the second portion 304 align or substantially align with the axes A-A and B-B of the first portion 302 (e.g., when the second portion 304 is inserted within the pocket 340) and when the first portion 302 is coupled to the subject's body, may be customized for the subject. More specifically, in view of FIG. 3C, in such an example, a first distance 317 (e.g., along the −X/+X axis of the coordinate axes of FIG. 3C, parallel to axis B-B) between vibrational device 316a and vibrational device 316b may correspond to a distance calculated between the first ischial tuberosity (IT) of the subject and the second ischial tuberosity (IT) of the subject. Similarly, in such an example, a second distance 319 (e.g., along the +Y/−Y axis of the coordinate axes of FIG. 3C, parallel to axis A-A) between the vibrational devices 316a, 316b and the vibrational device 316c may correspond to a distance calculated between the first and second ischial tuberosity (ITs) and the sacrum of the subject. Notably in such an aspect, since the second portion 304 is a reusable portion 300′, such customization may be beneficial to the subject. Similarly, according to another example, average distances to or between one or more specific pinpoint locations of a population of subject bodies (e.g., gender-based, height-based, weight-based, ethnicity-based, and/or the like) may be calculated, and a second portion 304, including one or more vibrational devices positioned or located within the second portion 304 to interface or correspond with the one or more specific pinpoint locations when the axes A′-A′ and B′-B′ of the second portion 304 align or substantially align with the axes A-A and B-B of the first portion 302 (e.g., when the second portion 304 is inserted within the pocket 340) and when the first portion 302 is coupled to a subject's body, may be customized for a subject within that population of subjects. According to various embodiments described herein, each vibrational device 316a, 316b, 316c may be a low-profile vibrational motor. In view of FIG. 3A, low-profile may correspond to a thickness 321 capable of being absorbed or padded by the second material of the second portion 304, the first material of the first portion 302 and/or the first material or different material of the pocket 340. For example, the thickness 321 of each vibrational device 316a, 316b, 316c may be less than the second portion thickness 311, the first portion thickness 309 and/or the pocket thickness 329. According to other aspects, low-profile may correspond to a thickness 321 such that a subject may be unable to noticeably feel, detect, or observe the presence (when the second portion 304 is inserted in the pocket 340 of the first portion 302) of each vibrational device (e.g., 316a, 316b, 316c) when seated or lying down. Such a low-profile may minimize and/or avoid unnecessary pressure points within the target treatment location and/or enable a low-profile system 300 (e.g., a low-profile sacral dressing). According to various embodiments of the present disclosure, each vibrational device 316a, 316b, 316c may be configured to, when activated, provide vibrations (e.g., vibrational energy), as described herein, through the second material of the second portion 304 and/or the first material of the first portion 302 (when inserted in the pocket 340) to the target treatment location (e.g., sacrum, ITs, and/or the like) to promote increased perfusion and an increased rate of healing.

Referring again to FIG. 3A, the control system 318 of the second portion 304, similar to the device 100 of FIGS. 1A and 1B and the device 200 of FIGS. 2A and 2B, may include a control device 320 and a power device 322. The control device 320 may be electrically coupled to the power device 322 (e.g., via a wire or the like). Notably, the control device 320 may include the control device 120 as structurally and functionally described in FIG. 1A herein (shown in FIG. 3A), the control device 220 as structurally and functionally described in FIG. 2A herein, and/or the like. In either case, the control device 320 may be configured to activate each vibrational device 316a, 316b, 316c. According to various embodiments, the control device 320 may be configured to activate each vibrational device 316a, 316b, 316c simultaneously, substantially simultaneously, individually, and/or intermittently, as described herein.

Further in such aspects, similar to the device 100 of FIGS. 1A and 1B and the device 200 of FIGS. 2A and 2B, the power device 322 may be configured to power the plurality of vibrational devices (e.g., 316a, 316b, 316c). Such a power device 322 may be positioned within the second portion 304 at a predetermined location that avoids spatial interference with the plurality of vibrational devices 316a, 316b, 316c (e.g., to minimize and/or avoid unnecessary pressure points within the target treatment location and/or to enable a low-profile second portion 304 to enable a low-profile system 300). The power device 322 may have a low-profile. Referring to FIG. 3A, low-profile may correspond to a thickness 327 capable of being absorbed or padded by the second material of the second portion 304, the first material of the first portion 302, and/or the first material or different material of the pocket 340. For example, the thickness 327 of the power device 322 may be less than the second portion thickness 111, the first portion thickness 309 and/or the pocket thickness 329. According to other aspects, low-profile may correspond to a thickness 327 such that a subject may be unable to noticeably feel, detect, or observe the presence (when the second portion 304 is inserted in the pocket 340 of the first portion 302) of the power device 322 when seated or lying down. According to various embodiments, the power device 322 may be configured to power the plurality of vibrational device for a predetermined period of time. According to some embodiments, the power device 322 may include a wireless power receiver 328 configured to wirelessly power, wirelessly charge, and/or wirelessly recharge the power device 322. In one example, the wireless power receiver 328 may include a reception area having electromagnetic receiving portion (not shown). According to such aspects, the reception area of the wireless power receiver 328 may be placed in proximity to and/or in alignment with an active area of an electromagnetic emitting portion (e.g., inductive coils generating an electromagnetic field) of a wireless power transmitter (not shown) to transfer power to and wirelessly power, wirelessly charge, and/or wirelessly recharge the power device 322. Here, according to various aspects, since the second portion 304 is removable, the second portion may be removed from the first portion 302 (e.g., while the first portion 302 is still adhered to the subject) to place the reception area of its wireless power receiver 32 in proximity to and/or alignment with the active area of the wireless power transmitter to transfer power to and wirelessly power, wirelessly charge, and/or wirelessly recharge the power device 322. According to further embodiments, the power device 322 may include an externally accessible power port 332 to power, charge, and/or recharge the power device 222 (e.g., via an AC to DC power supply, and/or the like). According to various aspects, the power device 322 may include a battery. According to some aspects, the power device 322 may include a rechargeable battery. According to alternative aspects, the power device 322 may include an externally accessible power port 332 to power the plurality of vibrational devices (e.g., 316a, 316b, 316c) with an external power source when a power source internal to the second portion 304 is not present or is depleted. According to some aspects, the power device 322 may include an alert device 354 (e.g., beeper, indicator light, wireless transmitter, and/or the like) configured to signal (e.g., audibly, visually, wirelessly, and/or the like) that the power device 322 is depleted or near depletion. For example, the alert device 354 (e.g., wireless transmitter) may transmit a signal to a wireless receiver (e.g., a cell phone, a user/subject device, a caregiver device, and/or the like) that the power device 322 is depleted or near depletion (e.g., to prompt replacement of the sacral dressing, to prompt a charge/recharge of the power device 322, and/or the like).

FIG. 4 depicts a plan view of an illustrative vibrational dressing device 400 that applies a low-intensity vibration and heat to a target treatment location according to one or more embodiments of the present disclosure. Referring to FIG. 4, for example, the device 100 of FIGS. 1A and 1B, as described herein, may be modified to include a skin warming unit 436 and a heat switch device 434.

In view of FIG. 4, according to various aspects, the skin warming unit 436 may include a flat heating element/coil arrangement. More specifically, the skin warming unit 436 may include a first heating coil 438 that surrounds vibrational device 116a′, a second heating coil 440 that surrounds vibrational device 116b′, and a third heating coil 442 that surrounds vibrational device 116c′. According to various aspects, the first heating coil 438 may surround vibrational device 116a′, the second heating coil 440 may surround vibrational device 116b′, and the third heating coil 442 may surround vibrational device 116c′ to focus heat at the target treatment location (e.g., sacrum, TIs, and/or the like). According to an alternative embodiment (not shown) the skin warming unit may cover an entire bottom surface of the second portion 104′ or a portion thereof. According to yet a further embodiment, similar skin warming unit may cover all or a portion of the bottom surface of the first portion 102′. According to various embodiments of the present disclosure, the skin warming unit 436 is configured to apply heat to the target treatment location to further promote perfusion and healing.

Referring still to FIG. 4, the control system 118′ of the second portion 104′ may include a control device 120′ and a power device 122′. The control device 120′ may be electrically coupled to the power device 122′ (FIG. 4, e.g., via a wire or the like). In such aspects, the control device 120′ may be configured (e.g., via firmware, hardware, software, and/or the like) to activate each vibrational device 116a′, 116b′, 116c′ (as described herein) and to activate the first heating coil 438, the second heating coil 440, and the third heating coil 442 of the skin warming unit 436. In view of FIG. 4, according to various aspects, the control device 120′ may be electrically coupled, via wire terminals 446 and/or 448 (e.g., wires depicted as dashed lines in FIG. 4), to the vibrational devices 116a′, 116b′, 116c′. According to an alternative aspect, the control device 120′ may be electrically coupled to each vibrational device 116a′, 116b′, 116c′ individually (not shown). The control device 120′ may also be electrically coupled to the first heating coil 438, the second heating coil 440, and the third heating coil (e.g., wires depicted as dashed lines in FIG. 4). According to various aspects described herein, the control device 120′ may include a vibration switch device 130′, the heat switch device 434, a processor 124′ and a memory 126′, where the memory 126′ stores program instructions executable by the processor 124′ to activate each vibrational device 116a′, 116b′, 116c′ (as described herein) and to activate the first heating coil 438, the second heating coil 440, and the third heating coil 442 of the skin warming unit 436.

According to various aspects, a first terminal (e.g., negative terminal) of the power device 122′ may be electrically coupled to each vibrational device 116a′, 116b′, 116c′ (FIG. 4, e.g., at wire terminal 444) and the skin warming unit 436 (e.g., wires depicted as dashed lines in FIG. 4) while a second terminal (e.g., positive terminal) of the power device 122′ may be electrically coupled to the vibration switch device 130′ and the heat switch device 434. In such an aspect, a circuit between the processor 124′ and the power device 122′ may be selectively interrupted by the vibration switch device 130′. Similarly, a circuit between the processor 124′ and the power device 122′ may be selectively interrupted by the heat switch device 434. According to various aspects, the vibration switch device 130′ may include a pull tab, where after the pull tab is removed the circuit between the processor 124′ and the power device 122′ is established to activate each vibrational device 116a′, 116b′, 116c′ (as described herein). Similarly, the heat switch device 434 may include a pull tab, where after the pull tab is removed the respective circuit between the processor 124′ and the power device 122′ is established to activate the first heating coil 438, the second heating coil 440, and the third heating coil 442 of the skin warming unit 436. According to other aspects, the vibration switch device 130′ may include a mechanical switch (e.g., a slide switch, on/off switch, and/or the like) configured to selectively establish, disconnect, and/or re-establish the circuit between the processor 124′ and the power device 122′. Similarly, the heat switch device 434 may include a mechanical switch (e.g., a slide switch, on/off switch, and/or the like) configured to selectively establish, disconnect, and/or re-establish the circuit between the processor 124′ and the power device 122′. According to numerous aspects the vibration switch device 130′ and/or the heat switch device 434 may be externally accessible on or within the second portion 104′. For example, in one embodiment (e.g., disposable device) of the present disclosure, a user may remove a pull tab to activate (e.g., via the processor 124′ executing the program instructions) each vibrational device 116a′, 116b′, 116c′ (e.g., on demand) before, during, or after the device 400 is applied to the target treatment location. Similarly, a user may remove a pull tab to activate (e.g., via the processor 124′ executing the program instructions) the first heating coil 438, the second heating coil 440, and the third heating coil 442 of the skin warming unit 436 (e.g., on demand) before, during, or after the device 400 is applied to the target treatment location. In another example, a user may selectively activate (e.g., via the processor 124′ executing the program instructions) each vibrational device 116a′, 116b′, 116c′ by actuating the vibration switch device 130′ (e.g., to an “on” position). Similarly, a user may selectively activate (e.g., via the processor 124′ executing the program instructions) the first heating coil 438, the second heating coil 440, and the third heating coil 442 of the skin warming unit 436 by actuating the heat switch device 434 (e.g., to an “on” position). According to various aspects, the vibration switch device 130′ and/or the heat switch device 434 may be configured to avoid an accidental actuation (e.g., from the “off” to the “on” position and/or from the “on” to the “off” position). In some aspects, the vibration switch device 130′ and/or the heat switch device 434 may include a locking mechanism (not shown) configured to lock the vibration switch device 130′ and heat switch device 434, respectively, in the “on” position and/or the “off” position. In other aspects, the vibration switch device 130′ and/or the heat switch device 434 may be configured such that it is not actuated by an interface pressure (e.g., due to subject weight) between the second portion 104′ (e.g., and/or components thereof) and the target treatment location of the subject and/or an external surface (e.g., a chair, a hospital bed, and/or the like). According to some aspects, the application of heat may be contraindicated with respect to the prevention of a pressure injury. For example, it may be desired to keep subject skin cool if warming the subject's skin would increase its metabolic demand and render it likely to break down. Accordingly, the heat switch device 434 enables selective application of heat when desired for increased perfusion and wound healing if the nature of the wound permits.

Further in such aspects, the power device 122′ may be configured to power the plurality of vibrational devices (e.g., 116a′, 116b′, 116c′) and the first heating coil 438, the second heating coil 440, and the third heating coil 442 of the skin warming unit 436. Such a power device 122′ may be positioned within the second portion 104′ at a predetermined location that avoids spatial interference with the plurality of vibrational devices (e.g., to minimize and/or avoid unnecessary pressure points within the target treatment location and/or to enable a low-profile device as described herein) and the skin warming unit 436 (e.g., to avoid overheating). The power device 122′ may have a low-profile (e.g., FIG. 1B). According to various aspects, low-profile may correspond to a thickness capable of being absorbed or padded by the second material of the second portion 104′ and/or the first material of the first portion 102′. According to other aspects, low-profile may correspond to a thickness such that a subject may be unable to noticeably feel, detect, or observe the presence of the power device 122′ when seated or lying down. According to various embodiments, the power device 122′ may be configured to power the plurality of vibrational devices and/or the skin warming unit 436 for a predetermined period of time. According to some embodiments, the power device 122′ may include a wireless power receiver 128′ configured to wirelessly power, wirelessly charge, and/or wirelessly recharge the power device 122′. In one example, the wireless power receiver 128′ may include a reception area having electromagnetic receiving portion (not shown). According to such aspects, the reception area of the wireless power receiver 128′ may be placed in proximity to and/or in alignment with an active area of an electromagnetic emitting portion (e.g., inductive coils generating an electromagnetic field) of a wireless power transmitter (not shown) to transfer power to and wirelessly power, wirelessly charge, and/or wirelessly recharge the power device 122′. According to further embodiments, the power device 122′ may include an externally accessible power port 132′ to power, charge, and/or recharge the power device 122′ (e.g., via an AC to DC power supply, and/or the like). According to various aspects, the power device 122′ may include a battery. According to some aspects, the power device 122′ may include a rechargeable battery. According to alternative aspects, the power device 122′ may an externally accessible power port 132′ to power the plurality of vibrational devices (e.g., 116a, 116b, 116c) and/or the first heating coil 438, the second heating coil 440, and the third heating coil 442 of the skin warming unit 436 with an external power source when a power source internal to the second portion 104′ is not present or depleted. It should be understood that the skin warming unit 436 described above may be similarly integrated within the device 200 of FIG. 2 and/or the second portion 304 of the system 300 of FIG. 3C including the reusable portion 300′ and the disposable portion 300″ of FIGS. 3A and 3B.

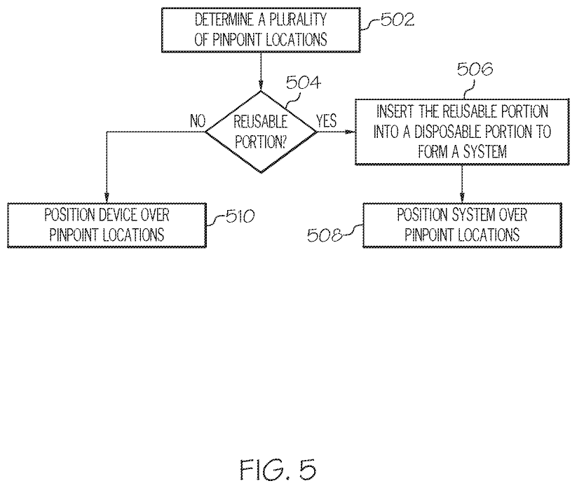

Referring to FIG. 5, various aspects of the present disclosure further include methods of placing the vibrational dressing devices and/or the vibrational dressing system, as described herein, on the subject. At block 502, a plurality of pinpoint locations may be determined. According to various aspects, a plurality of pinpoint locations on a subject's body may be determined (e.g., a pinpoint location of a first ischial tuberosity of the subject, a pinpoint location of a second ischial tuberosity of the subject, a pinpoint location of a sacrum of the subject, and/or the like). At block 504, it may be determined whether a vibrational dressing device (e.g., FIGS. 1A-1B, 2A-2B, 4) or a vibrational dressing system (e.g., FIG. 3C) is to be placed on the subject. More specifically, at block 504, it may be determined whether the vibrational dressing includes a reusable portion (e.g., FIG. 3A). In one example, if the vibrational dressing includes more than one separate portion (e.g., FIGS. 3A and 3B) and one portion includes a pocket (e.g., FIG. 3B, pocket 340) and the other portion lacks a bonding portion (e.g., FIG. 3A, lacks a bonding portion 306 of FIG. 3B), then that vibrational dressing may include a reusable portion (e.g., FIG. 3A) of a vibrational dressing system (e.g., FIG. 3C). In another example, if the vibrational dressing does not include more than one separate portion and the vibrational dressing lacks a bonding portion (e.g., FIG. 3A, lacks a bonding portion 306 of FIG. 3B), then that vibrational dressing may include a reusable portion (e.g., FIG. 3A) of a vibrational dressing system (e.g., FIG. 3C). In yet another example, if the vibrational dressing does not include more than one separate portion, the vibrational dressing includes a bonding portion (e.g., FIG. 1B includes bonding portion 106, FIG. 2B includes bonding portion 206), and the vibrational dressing does not include a pocket (e.g., FIG. 3B, pocket 340), then that vibrational dressing may not include a reusable portion (e.g., FIG. 3A) and may be a vibrational dressing device (e.g., FIGS. 1A-1B, 2A-2B, 4). At block 506, if the vibrational dressing includes a reusable portion, the reusable portion may be inserted into a disposable portion to form a system (e.g., vibrational dressing system). At block 508, the system may be positioned over the pinpoint locations. According to various aspects, the system may be positioned over the plurality of determined pinpoint locations. According to further aspects, the system (e.g., vibrational dressing system) may be positioned on the subject such that each of a plurality of vibrational devices associated with the system (e.g., vibrational dressing system) is located over each determined pinpoint location of the subject's body (e.g., the pinpoint location of a first ischial tuberosity of the subject, the pinpoint location of a second ischial tuberosity of the subject, the pinpoint location of a sacrum of the subject, and/or the like). Alternatively, at block 510, if the vibrational dressing does not include a reusable portion, a device may be positioned over the pinpoint locations. According to various aspects, the device may be positioned over the plurality of determined pinpoint locations. According to further aspects, the device (e.g., vibrational dressing device) may be positioned on the subject such that each of a plurality of vibrational devices associated with the device (e.g., vibrational dressing device) is located over each determined pinpoint location of the subject's body (e.g., the pinpoint location of a first ischial tuberosity of the subject, the pinpoint location of a second ischial tuberosity of the subject, the pinpoint location of a sacrum of the subject, and/or the like).

As such, in one example, a method for placing a vibrational dressing device on a subject may include determining a location of a first ischial tuberosity of the subject's body, a second ischial tuberosity of the subject's body, and a sacrum of the subject's body, and positioning a first vibrational device of the vibrational dressing over the first ischial tuberosity of the subject's body, a second vibrational device of the vibrational dressing over the second ischial tuberosity of the subject's body, and a third vibrational device of the vibrational dressing over the sacrum of the subject' body. In another example, a method for placing a vibrational dressing system on a subject may include determining a location of a first ischial tuberosity of the subject's body, a second ischial tuberosity of the subject's body, and a sacrum of the subject's body, inserting a reusable second portion into a pocket of a disposable first portion to form the vibrational dressing system, and positioning the vibrational dressing system on the subject such that a first vibrational device of the reusable second portion is located over the first ischial tuberosity of the subject's body, a second vibrational device of the reusable second portion is located over the second ischial tuberosity of the subject's body, and a third vibrational device of the reusable second portion is located over the sacrum of the subject's body.

According to various embodiments of the present disclosure, the vibrations (e.g., vibrational energy) provided to the target treatment location (e.g., sacrum, ITs, and/or the like) by the one or more vibrational device, as described herein, may include low-intensity vibration. According to various aspects, such low-intensity vibration may increase skin blood flow both during and after the application of such vibrations (e.g., vibrational energy) and may increase the rate of wound healing. According to other aspects, periodic (e.g., daily, hourly, and/or the like) low-intensity vibration may increase the rate of wound closure and re-epithelialization (e.g., the covering of a skin wound with a new epithelium). According to yet further aspects, low-intensity vibration may reduce edema (e.g., swelling) which may decrease interstitial pressure and improve microcirculation to allow substances (e.g., nutrients) to access a wound area. According to yet other aspects, low-intensity vibration may promote angiogenesis (e.g., formation/growth of new blood vessels from pre-existing blood vessels) and promote granulation tissue (e.g., formation/growth of new connective tissue and microscopic blood vessels on the surfaces of a wound during healing). According to some aspects, low-intensity vibration may exert a mechanical stress effect on tissue to promote cell proliferation (e.g., increase in cells due to cell growth and cell division). More specifically, such mechanical stresses may increase the expression of growth factors and increase blood flow at the target treatment location.

Referring to FIGS. 1A, 2A, 3C, and 4, each control device 120, 220, 320, and 120′ respectively may activate a plurality of vibrational devices, as described herein. With respect to FIGS. 1A, 2A, 3C, and 4, according to various aspects, each control device 120, 220, 320, and 120′ respectively may be configured (e.g., via firmware, hardware, software, and/or the like) to activate the plurality of vibrational devices with a low-intensity vibration. According to some aspects, each control device 120, 220, 320, 120′ may include a signal generator 152, 252, 352, 152′ to generate and transmit a signal including at least one waveform (e.g., square, sinusoidal, pulse, triangle, and/or the like) where the frequency of the signal is from about 30 Hz to about 90 Hz and the signal causes the plurality of vibrational devices, as described herein, to produce vibrations having a peak acceleration from about 0.2 g to about 0.4 g (e.g., measurable via an accelerometer or the like). According to some aspects, an accelerometer may be coupled (e.g., permanently or temporarily) to or embedded within the second portion 104 of device 100, the second portion 204 of device 200, the second portion 304 of system 300, and/or the second portion 104′ of device 400 to calibrate and/or confirm the peak acceleration of the vibrations being produced via the plurality of vibrational devices. According to other aspects, an accelerometer may be embedded within each of the plurality of vibrational devices to calibrate and/or confirm the peak acceleration of the vibrations being produced by each of the plurality of vibrational devices. In one example, the generated signal may include a waveform where the frequency is about 45 Hz and the signal causes the plurality of vibrational devices to produce vibrations having a peak acceleration of about 0.4 g. According to other aspects, each control device 120, 220, 320, 120′ may include a signal generator 152, 252, 352, 152′ to generate and transmit a signal including at least one waveform (e.g., square, sinusoidal, pulse, triangle, and/or the like) where the frequency of the signal is from about 30 Hz to about 50 Hz and the signal causes the plurality of vibrational devices, as described herein, to produce vibrations having a peak acceleration from about 0.2 g to about 0.4 g. For example, the generated signal may include a waveform where the frequency is about 30 Hz and the signal causes the plurality of vibrational devices to produce vibrations having a peak acceleration of about 0.4 g (e.g., corresponding to an amplitude of 0.2 mm). According to alternative aspects, each control device 120, 220, 320, 120′ may include a signal generator 152, 252, 352, 152′ to generate and transmit a signal including at least one waveform (e.g., square, sinusoidal, pulse, triangle, and/or the like) where the frequency of the signal is from about 30 Hz to about 50 Hz and the signal causes the plurality of vibrational devices, as described herein, to produce vibrations having a peak acceleration from about 6 g to about 7 g (e.g., corresponding to an amplitude of about 5 mm to about 6 mm). According to further alternative aspects, each control device 120, 220, 320, 120′ may include a signal generator 152, 252, 352, 152′ to generate and transmit a signal including at least one waveform (e.g., square, sinusoidal, pulse, triangle, and/or the like) where the frequency of the signal is from about 1 Hz to about 15 kHz and the signal causes the plurality of vibrational devices, as described herein, to produce vibrations having a force from about 0.001 N to about 100 N (e.g., corresponding to an amplitude of about 1 μm to about 15 mm). According to various aspects, Such vibrational forces may be calculated (e.g., via Newton's second law of motion, F=ma) using an accelerometer coupled to or embedded within the second portion 104, 204, 304, and/or 104′ and/or embedded within each of the vibrational devices, as described herein.

Further with respect to FIGS. 1A, 3A, and 4, according to some aspects, each control device 120, 320 and 120′ respectively may include a memory that stores program instructions executable by a processor (as described above) to activate the plurality of vibrational devices with a low-intensity vibration. In such aspects, each processor may send a control signal to its respective signal generator 152, 352, 152′ to generate and transmit the signals as described above.

According to various embodiments of the present disclosure, the vibrations (e.g., vibrational energy) provided to the target treatment location (e.g., sacrum, ITs, and/or the like) by the one or more vibrational device, as described herein, may include scheduled vibration.

Referring again to FIGS. 1A, 3A, and 4, each control device 120, 320, and 120′ respectively may be configured to activate a plurality of vibrational devices, as described herein. More specifically, according to various aspects, each control device 120, 320, and 120′ respectively may be configured (e.g., via firmware, hardware, software, and/or the like) to activate the plurality of vibrational devices, as described herein, according to a predefined schedule. Further, according to some aspects, each control device 120, 320 and 120′ respectively may include a memory that stores program instructions executable by a processor (as described above) to activate the plurality of vibrational devices, as described herein, according to a predefined schedule. In such aspects, the predefined schedule may include one or more set time (e.g., a specific time of day, specifically spaced-apart times during a day, and/or the like) for a set duration (e.g., seconds, minutes, hours, and/or the like) of vibrations (e.g., vibrational energy) over a predetermined period (e.g., an hour, a day, a week, and/or the like). For example, the predefined schedule may apply vibrations (e.g., via one or more of the plurality of vibrational devices) to the target treatment location every “X” hours for “Y” minutes for “Z” days, where each of “X”, “Y”, and “Z” may be the same or different. As another example, the predefined schedule may apply vibrations to the target treatment location at “X” a.m. each day for “Y” minutes for “Z” days each week, where each of “X”, “Y”, and “Z” may be the same or different. According to another aspect, the predefined schedule may include one or more set duration (e.g., seconds, minutes, hours, and/or the like) of vibrations (e.g., vibrational energy) over a predetermined period (e.g., an hour, a day, a week, and/or the like). For example, the predefined schedule may apply vibrations to the target treatment location for “X” minutes per “Y” day(s), where each of “X” and “Y” may be the same or different. According to further aspects, in line with above, the predefined schedule may further associate and apply a same or a different intensity of vibrational energy (via the one or more of the plurality of vibrational devices) during each application of vibrations.

According to various embodiments of the present disclosure, the vibrations (e.g., vibrational energy) provided to the target treatment location (e.g., sacrum, ITs, and/or the like) by the one or more vibrational device, as described herein, may include intermittent vibration.

Referring again to FIGS. 1A, 3A, and 4, each control device 120, 320, and 120′ respectively may be configured to activate a plurality of vibrational devices, as described herein. More specifically, according to various aspects, each control device 120, 320, and 120′ respectively may be configured (e.g., via firmware, hardware, software, and/or the like) to activate the plurality of vibrational devices intermittently. Further, according to some aspects, each control device 120, 320 and 120′ respectively may include a memory that stores program instructions executable by a processor (as described above) to activate the plurality of vibrational devices intermittently. In such aspects, intermittent vibration may include irregular, varying and/or random vibrations of a duration (e.g., seconds, minutes, hours, and/or the like) of vibrations (e.g., vibrational energy) over a predetermined period (e.g., an hour, a day, a week, and/or the like). For example, the intermittent vibration may apply vibrations (e.g., via one or more of the plurality of vibrational devices) to the target treatment location for “V” minutes, then taper the intensity of the vibrational energy for “W” minutes, then remove/stop vibrations for “X” minutes, and then ramp the intensity of the vibrational energy for “Y” minutes, where such applications may occur randomly for “Z” hours, and where each of “V”, “W”, “X”, “Y” may be the same or different. According to another aspect, intermittent vibration may include an alternating sequence of vibrations having one or more durations (e.g., seconds, minutes, hours, and/or the like) of “on” time and one or more durations (e.g., seconds, minutes, hours, and/or the like) of “off” time over a predetermined period (e.g., an hour, a day, a week, and/or the like). For example, the intermittent vibration may apply vibrations (e.g., via one or more of the plurality of vibrational devices) to the target treatment location for “X” minutes, and then remove/stop vibrations for “Y” minutes, where such application and removal of vibrations may occur/repeat for “Z” hours, and where each of “X”, “Y” and “Z” may be the same or different. According to further aspects, in line with above, the intermittent vibration may further associate and apply a same or a different intensity of vibrational energy (via the one or more of the plurality of vibrational devices) during each application of vibrations. It should now be understood that the systems, devices, and methods described herein deliver low-intensity vibrations to a target treatment location of a subject via a specific arrangement of a plurality of vibrational devices. In particular, each vibrational device may be positioned to correspond with a specific location on a body of the subject within the target treatment location when coupled to the body of the subject. Such low-intensity vibrations may increase skin blood flow both during and after the application of such vibrations and may increase the rate of chronic wound healing.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A vibrational dressing device, comprising:
a first portion contoured to a target treatment location of a subject; and
a second portion coupled to the first portion, the second portion comprising:
a plurality of vibrational devices that vibrate the target treatment location, wherein each vibrational device is positioned within the second portion to correspond with a particular location on a body of the subject within the target treatment location when the first portion is coupled to the body of the subject; and
a control system to selectively activate one or more of the plurality of vibrational devices.

2. The vibrational dressing device of claim 1, wherein the plurality of vibrational devices include a first vibrational device, a second vibrational device, and a third vibrational device, and wherein the first vibrational device is located within the second portion to correspond with a first ischial tuberosity of the subject, the second vibrational device is located within the second portion to correspond with a second ischial tuberosity of the subject, and the third vibrational device is located within the second portion to correspond with a sacrum of the subject.

3. The vibrational dressing device of claim 1, wherein the first portion is contoured to a sacrum of the subject.

4. The vibrational dressing device of claim 1, wherein the control system comprises:
a vibration switch; and
a signal generator that transmits a signal to the plurality of vibrational devices, the signal causing the plurality of vibrational devices to generate low-intensity vibrations.

5. The vibrational dressing device of claim 4, wherein the signal causes the plurality of vibrational devices to generate vibrations having a peak acceleration from about 0.2 g to about 0.4 g.

6. The vibrational dressing device of claim 4, wherein the control system activates the plurality of vibrational devices according to a predefined schedule.

7. The vibrational dressing device of claim 4, wherein the control system further comprises a power device, and wherein the power device includes a wireless power receiver to wirelessly power, wirelessly charge, or wirelessly recharge the power device.

8. The vibrational dressing device of claim 4, wherein the control system further comprises a processor and a memory, and wherein the memory stores program instructions executable by the processor to send a control signal to the signal generator, the control signal causing the signal generator to generate and to transmit the signal to the plurality of vibrational devices.

9. The vibrational dressing device of claim 4, wherein the control system further comprises a processor and a memory, and wherein the memory stores program instructions executable by the processor to activate the plurality of vibrational devices at least one of simultaneously, individually, or intermittently.

10. The vibrational dressing device of claim 1, wherein the first portion includes a bonding portion that releasably couples the first portion to the body of the subject.

11. The vibrational dressing device of claim 10, wherein the bonding portion extends a first width inwardly from a peripheral outer edge of the first portion around a perimeter of the first portion.

12. A vibrational dressing device, comprising:
a first portion; and
a second portion coupled to the first portion, the second portion comprising:
a plurality of vibrational devices that vibrate a target treatment location, the plurality of vibrational devices arranged within the second portion as a predefined array of vibrational devices; and
a control system to selectively activate the plurality of vibrational devices.

13. The vibrational dressing device of claim 12, wherein the first portion is at least one of shaped or sized to contour to the target treatment location, the target treatment location including a first ischial tuberosity of the subject, a second ischial tuberosity of the subject, and a sacrum of the subject.

14. The vibrational dressing device of claim 12, wherein the control system activates the plurality of vibrational devices according to a predefined schedule.

15. A vibrational dressing system, comprising:
a disposable first portion; and
a reusable second portion, comprising:
a plurality of vibrational devices, wherein each vibrational device is positioned within the reusable second portion to correspond with a particular location on a body of a subject within a target treatment location; and
a control system, comprising:
a vibration switch; and
a signal generator that transmits a signal to the plurality of vibrational devices, the signal causing the plurality of vibrational devices to generate low-intensity vibrations.

16. The vibrational dressing system of claim 15, wherein the disposable first portion is contoured to the target treatment location of the subject, the target treatment location including a first ischial tuberosity of the subject, a second ischial tuberosity of the subject, and a sacrum of the subject.

17. The vibrational dressing system of claim 15, wherein the plurality of vibrational devices are arranged within the reusable second portion as a predefined array of vibrational devices.

18. The vibrational dressing system of claim 17, wherein the predefined array of vibrational devices is based on at least one distance calculated between one or more pinpoint location of the body of the subject.

19. The vibrational dressing system as in claim 15, wherein at least one of the disposable first portion or the reusable second portion comprises a skin warming unit to selectively heat the target treatment location.

20. The vibrational dressing system of claim 19, wherein the skin warming unit comprises a coil that surrounds each of the plurality of vibrational devices to focus heat to the target treatment location.
